(19) United States
(12) Patent Application Publication
(10) Pub. No.: US 2006/0212077 A1
(43) Pub. Date: Sep. 21, 2006

(54) Electromagnetic treatment apparatus for augmenting wound repair and method for using same

(76) Inventors: Arthur A. Pilla, Oakland, NJ (US); Andre DiMino, Woodcliff Lake, NJ (US)

Correspondence Address: LEN TAYLOR, PATENT ATTORNEY, 261 DAVENPORT STREET, SOMERVILLE, NJ, 08876, US

(21) Appl. No.: 11/369,308

(22) Filed: Mar. 6, 2006

Related U.S. Application Data
(60) Provisional application No. 60/658,967, filed on Mar. 7, 2005.

(51) Int. Cl. A61N 1/00; A61K 38/22

(52) U.S. Cl. 607/2; 514/12

(57) ABSTRACT

A method for augmenting acute and chronic wound repair comprising the steps of configuring at least one waveform having at least one waveform parameter, selecting a value of said at least one waveform parameter of said at least one waveform to maximize at least one of a signal to noise ratio and a Power signal to noise ratio, in a target pathway structure, using said at least one waveform that maximizes said at least one of a signal to noise ratio and a Power signal to noise ratio in a target pathway structure, to generate an electromagnetic signal, and coupling said electromagnetic signal to said target pathway structure to accelerate healing mechanisms.

This application claims the benefit of U. S. Provisional Application No. 60/658,967 filed Mar. 7, 2005.

### BACKGROUND OF THE INVENTION

1. Field of the Invention

This invention relates to augmenting wound repair in humans, plants, and animals by altering the interaction with the electromagnetic environment of living tissues, cells, and molecules. The invention also relates to a method of modification of cellular and tissue growth, repair, maintenance and general behavior by the application of encoded electromagnetic information. More particularly, this invention provides for an application of highly specific electromagnetic frequency (“EMF”) signal patterns to one or more body parts by surgically non-invasive reactive coupling of encoded electromagnetic information. Such application of electromagnetic waveforms to human, animal, and plant target pathway structures such as cells, organs, tissues and molecules, can serve to enhance wound repair.

The use of most low frequency EMF has been in conjunction with applications of bone repair and healing. As such, EMF waveforms and current orthopedic clinical use of EMF waveforms comprise relatively low frequency components and are of a very low power, inducing maximum electrical fields in a millivolts per centimeter (mV/cm) range at frequencies under five KHz. A linear physicochemical approach employing an electrochemical model of cell membranes to predict a range of EMF waveform patterns for which bioeffects might be expected is based upon an assumption that cell membranes, and specifically ion binding at structures in or on cell membranes, are a likely EMF target. Therefore, it is necessary to determine a range of waveform parameters for which an induced electric field could couple electrochemically at a cellular surface, such as by employing voltage-dependent kinetics. Extension of this linear model involves Lorentz force considerations that eventually demonstrated that the magnetic component of EMF could play a significant role in EMF therapeutics. This led to the ion cyclotron resonance and quantum models that predicts benefits from combined AC and DC magnetic field effects at very low frequency ranges.

A pulsed radio frequency (“PRF”) signal derived from a 27.12 MHz continuous sine wave used for deep tissue healing is known in the prior art of diathermy. A pulsed successor of the diathermy signal was originally reported as an electromagnetic field capable of eliciting a non-thermal biological effect in the treatment of infections. Subsequently, PRF therapeutic applications have been reported for the reduction of post-traumatic and post-operative pain and edema in soft tissues, wound healing, burn treatment, and nerve regeneration. The application of PRF for resolution of traumatic edema has become increasingly used in recent years. Results to date using PRF in animal and clinical studies suggest that edema may be measurably reduced from such electromagnetic stimulus.

The within invention is based upon biophysical and animal studies that attribute effectiveness of cell-to-cell communication on tissue structures' sensitivity to induced voltages and associated currents. A mathematical analysis using at least one of a Signal to Noise Ratio (“SNR”) and a Power Signal to Noise Ratio (“Power SNR”) evaluates whether EMF signals applied to target pathway structures such as cells, tissues, organs, and molecules, are detectable above thermal noise present at an ion binding location. Prior art of EMF dosimetry did not taken into account dielectric properties of tissue structures, rather the prior art utilized properties of isolated cells. By utilizing dielectric properties, reactive coupling of electromagnetic waveforms configured by optimizing SNR and Power SNR mathematical values evaluated, at a target pathway structure can enhance repair of various wounds in human, animal and plant cells, organs, tissues and molecules for example post-surgical and traumatic wound repair, angiogenesis, improved blood perfusion, vasodilation, vasoconstriction, edema reduction, enhanced neovascularization, bone repair, tendon repair, ligament repair, organ regeneration and pain relief. Wound repair enhancement results from increased blood flow and modulation of angiogenesis and neovascularization as well as from other enhanced bioeffective processes.

Recent clinical use of non-invasive PRF at radio frequencies has used pulsed bursts of a 27.12 MHz sinusoidal wave, each pulse burst typically exhibiting a width of sixty five microseconds and having approximately 1,700 sinusoidal cycles per burst, and with various burst repetition rates.

Broad spectral density bursts of electromagnetic waveforms having a frequency in the range of one to one hundred megahertz (MHz), with 1 to 100,000 pulses per burst, and with a burst-repetition rate of 0.01 to 10,000 Hertz (Hz), are selectively applied to human, animal and plant cells, organs, tissues and molecules. The voltage-amplitude envelope of each pulse burst is a function of a random, irregular, or other like variable, effective to provide a broad spectral density within the burst envelope. The variables are defined by mathematical functions that take into account signal to thermal noise ratio and Power SNR in specific target pathway structures. The waveforms are designed to modulate living cell growth, condition and repair. Particular applications of these signals include, but are not limited to, enhancing treatment of organs, muscles, joints, skin and hair, post surgical and traumatic wound repair, angiogenesis, improved blood perfusion, vasodilation, vasoconstriction, edema reduction, enhanced neovascularization, bone repair, tendon repair, ligament repair, organ regeneration and pain relief. The application of the within electromagnetic waveforms can serve to enhance healing of various wounds.

According to an embodiment of the present invention a pulse burst envelope of higher spectral density can more efficiently couple to physiologically relevant dielectric pathways, such as cellular membrane receptors, ion binding to cellular enzymes, and general transmembrane potential changes. An embodiment according to the present invention increases the number of frequency components transmitted to relevant cellular pathways, resulting in a larger range of biophysical phenomena applicable to known healing mechanisms becoming accessible, including enhanced enzyme activity, growth factor release and cytokine release. By increasing burst duration and by applying a random, or other high spectral density envelope, to a pulse burst envelope of mono- or bi-polar rectangular or sinusoidal pulses that induce peak electric fields between 10⁻⁶ and 10 volts per centimeter (V/cm), and that satisfy detectability requirements according to SNR or Power SNR, a more efficient and greater effect could be achieved on biological healing processes applicable to both soft and hard tissues in humans, animals and plants resulting in an acceleration of wound repair.

The present invention relates to known mechanisms of wound repair that involve the naturally timed release of the appropriate growth factor or cytokine in each stage of wound repair as applied to humans, animals and plants. Specifically, wound repair involves an inflammatory phase, angiogenesis, cell proliferation, collagen production, and remodeling stages. There are timed releases of specific cytokines and growth factors in each stage. Electromagnetic fields can enhance blood flow and enhance the binding of ions which, in turn, can accelerate each healing phase. It is the specific intent of this invention to provide an improved means to enhance the action of exogenous factors and accelerate repair. An advantageous result of using the present invention is that wound repair can be accelerated due to enhanced blood flow or enhanced biochemical activity. It is an object of the present invention to provide an improved means to accelerate the intended effects or improve efficacy as well as other effects of the cytokines and growth factors relevant to each stage of wound repair.

Another object of the present invention is to cause and accelerate healing of chronic wounds such as diabetic ulcers, venous stasis ulcers, pressure sores and non-healing wounds of any origin.

Another object of the present invention is that by applying a high spectral density voltage envelope as a modulating or pulse-burst defining parameter according to SNR and Power SNR requirements, power requirements for such increased duration pulse bursts can be significantly lower than that of shorter pulse bursts having pulses within the same frequency range; this results from more efficient matching of frequency components to a relevant cellular/molecular process. Accordingly, the advantages, of enhanced transmitted dosimetry to relevant dielectric pathways and of decreased power requirements are achieved.

Therefore, a need exists for an apparatus and a method that more effectively accelerates wound repair in human, animal and plant cells, organs, tissues and molecules. SUMMARY OF THE INVENTION

The present invention relates to accelerating wound repair of living tissues, cells and molecules by providing a therapeutic, prophylactic and wellness apparatus and method for non-invasive pulsed electromagnetic treatment to enhance condition, repair and growth of living tissue in animals, humans and plants. This beneficial method operates to selectively change a bio-electromagnetic environment associated with cellular and tissue environments by using electromagnetic means such as EMF generators and applicator heads. An embodiment according to the present invention comprises introducing a flux path to a selectable body region, comprising a succession of EMF pulses having a minimum width characteristic of at least 0.01 microseconds in a pulse burst envelope having between 1 and 100,000 pulses per burst, in which a voltage amplitude envelope of said pulse burst is defined by a randomly varying parameter in which an instantaneous minimum amplitude thereof is not smaller than a maximum amplitude thereof by a factor of one ten thousandth. Further, the repetition rate of such pulse bursts may vary from 0.01 to 10,000 Hertz. A mathematically definable parameter satisfying SNR and/or Power SNR detectability requirements in a target structure is employed to define the configuration of the pulse bursts.

It is another object of the present invention to provide a method of treating living cells and tissue by electromagnetically modulating sensitive regulatory processes at a cell membrane and at junctional interfaces between cells, using waveforms configured to satisfy SNR and Power SNR detectability requirements in a target pathway structure.

A preferred embodiment according to the present invention utilizes a Power Signal to Noise Ratio (“Power SNR”) approach to configure bioeffective waveforms and incorporates miniaturized circuitry and lightweight flexible coils. This advantageously allows a device that utilizes a Power SNR approach, miniaturized circuitry, and lightweight flexible coils, to be completely portable and if desired to be constructed as disposable and if further desired to be constructed as implantable.

Specifically, broad spectral density bursts of electromagnetic waveforms, configured to achieve maximum signal power within a bandpass of a biological target, are selectively applied to target pathway structures such as tissues, to enhance effectiveness of pharmacological, chemical, cosmetic and topical agents. Waveforms are selected using a unique amplitude/power comparison with that of thermal noise in a target pathway structure. Signals comprise bursts of at least one of sinusoidal, rectangular, chaotic and random wave shapes, have frequency content in a range of about 0.01 Hz to about 100 MHz at about 1 to about 100,000 bursts per second, and have a burst repetition rate from about 0.01 to about 1000 bursts/second. Peak signal amplitude at a target pathway structure such as organs, cells, tissues, and molecules, lies in a range of about 1 μV/cm to about 100 mV/cm. Each signal burst envelope may be a random function providing a means to accommodate different electromagnetic characteristics of enhancing bioeffective processes. A preferred embodiment according to the present invention comprises about 0.1 to about 100 millisecond pulse burst comprising about 1 to about 200 microsecond symmetrical or asymmetrical pulses repeating at about 0.1 to about 100 kilohertz within the burst. The burst envelope is a modified 1/f function and is applied at random repetition rates between about 0.1 and about 1000 Hz. Fixed repetition rates can also be used between about 0.1 Hz and about 1000 Hz. An induced electric field from about 0.001 mV/cm to about 100 mV/cm is generated. Another embodiment according to the present invention comprises an about 0.01 millisecond to an about 10 millisecond burst of high frequency sinusoidal waves, such as 27.12 MHz, repeating at about 1 to about 100 bursts per second. An induced electric field from about 0.001 mV/cm to about 100 mV/cm is generated. Resulting waveforms can be delivered via inductive or capacitive coupling.

It is an object of the present invention to provide modulation of electromagnetically sensitive regulatory processes at the cell membrane and at junctional interfaces between cells.

It is another object of the present invention to provide electromagnetic treatment for wound repair having a broad-band, high spectral density electromagnetic field configured according to at least one of SNR and Power SNR.

It is another object of the present invention to accelerate wound repair by configuring a power spectrum of a waveform by mathematical simulation by using signal to noise ratio (“SNR”) analysis to configure a waveform optimized to modulate angiogensis and neovascualarization, then coupling the configured waveform using a generating device such as ultra lightweight wire coils that are powered by a waveform configuration device such as miniaturized electronic circuitry.

It is another object of the present invention to modulate angiogenesis and neovascularization by evaluating Power SNR at any target pathway structure such as molecules, cells, tissues and organs to accelerate wound repair by using any input waveform, even if electrical equivalents are non-linear as in a Hodgkin-Huxley membrane model.

It is another object of the present invention to provide an apparatus that incorporates use of Power SNR in which amplitude modulation of the pulse burst envelope of the electromagnetic signal will induce coupling with a maximum number of relevant EMF-sensitive pathways in cells and tissues to enhance wound repair in humans, animals and plants.

It is another object of the present invention to provide a method and apparatus for enhancing wound repair using electromagnetic fields selected by optimizing a power spectrum of a waveform to be applied to a biochemical target pathway structure to enable modulation of angiogenesis and neovascularization within molecules, cells, tissues and organs.

It is another object of the present invention to significantly lower peak amplitudes and shorter pulse duration by matching via Power SNR, a frequency range in a signal to frequency response and sensitivity of a target pathway structure such as a molecule, cell, tissue, and organ thereby enabling modulation of angiogenesis and neovascularization for accelerating wound repair.

It is another object of the invention to provide a method of enhancing soft tissue and hard tissue repair.

It is another object of the invention to provide a method of increasing blood flow to affected tissues by modulating angiogenesis.

It is another object of the invention to provide an improved method of increasing blood flow to enhance the viability and growth or differentiation of implanted cells, tissues and organs.

It is another object of the invention to provide an improved method of increasing blood flow in cardiovascular diseases by modulating angiogenesis.

It is another object of the invention to provide beneficial physiological effects through improvement of micro-vascular blood perfusion and reduced transudation.

It is another object of the invention to provide an improved method of treatment of maladies of the bone and other hard tissue.

It is a still further object of the invention to provide an improved means of the treatment of edema and swelling of soft tissue.

It is another object to provide a means of repair of damaged soft tissue.

It is yet another object to provide a means of increasing blood flow to damaged tissue by modulation of vasodilation and stimulating neovascularization.

It is yet another object to enhance healing of post-surgical wounds by reducing the inflammatory phase and modulating growth factor release.

It is yet another object of the instant invention to reduce the inflammatory phase post-cosmetic surgery.

It is yet another object of the instant invention to reduce or eliminate the post-surgical complications of breast augmentation, such as capsular contractions.

It is yet another object of the instant invention to reduce post-surgical pain, edema and discoloration.

It is yet a further object of the present invention to treat chronic wounds such as diabetic ulcers, venous stasis ulcers, pressure sores and any non-healing wound with EMF signals configured according to an embodiment of the present invention.

It is a yet a further object to provide apparatus for use of an electromagnetic method of the character indicated, wherein operation of the apparatus can proceed at reduced power levels as compared to those of related methods known in electromedicine and respective biofield technologies, with attendant benefits of safety, economics, portability, and reduced electromagnetic interference.

It is a further object of the present invention to provide a method for treatment to enhance wellness.

It is a further object of the present invention to provide a method in which electromagnetic waveforms are configured according to SNR and Power SNR detectability requirements in a target pathway structure.

It is another object of the present invention to provide a method for electromagnetic treatment comprising a broadband, high spectral density electromagnetic field.

It is another object of the present invention to provide a method of enhancing soft tissue and hard tissue repair by using EMF.

It is another object of the present invention to provide a method to increase blood flow to affected tissues by using electromagnetic treatment to modulate angiogenesis.

It is yet a further object of the present invention to provide a method of treatment of chronic wounds such as diabetic ulcers, venous stasis ulcers, pressure sores and any non-healing wound.

It is another object of the present invention to provide a method to increase blood flow to regulate viability, growth, and differentiation of implanted cells, tissues and organs.

It is another object of the present invention to provide a method to treat cardiovascular diseases by modulating angiogensis and increasing blood flow.

It is another object of the present invention to provide a method to improve micro-vascular blood perfusion and reduce transudation.

It is another object of the present invention to provide a method to increase blood flow to treat maladies of bone and hard tissue.

It is another object of the present invention to provide a method to increase blood flow to treat edema and swelling of soft tissue.

It is another object of the present invention to provide a method to increase blood flow to repair damaged soft tissue.

It is another object of the present invention to provide a method to increase blood flow to damaged tissue by modulation of vasodilation and stimulating neovascularization.

It is a further object of the present invention to provide an electromagnetic treatment apparatus wherein the apparatus operates using reduced power levels.

It is a yet further object of the present invention to provide an electromagnetic treatment apparatus wherein the apparatus is inexpensive, portable, and produces reduced electromagnetic interference.
The above and yet other objects and advantages of the present invention will become apparent from the hereinafter set forth Brief Description of the Drawings, Detailed Description of the Invention, and Claims appended herewith.

### BRIEF DESCRIPTION OF THE DRAWINGS

Preferred embodiments of the present invention will be described below in more detail, with reference to the accompanying drawings:

FIG. 1 is a flow diagram of a method for accelerating wound repair in living tissues, cells and molecules according to an embodiment of the present invention;

FIG. 2 is a view of control circuitry and electrical coils applied to a knee joint according to a preferred embodiment of the present invention;

FIG. 3 is a block diagram of miniaturized circuitry according to a preferred embodiment of the present invention;

FIGS. 4A is a line drawing of a wire coil such as an inductor according to a preferred embodiment of the present invention;

FIGS. 4B is a line drawing of a flexible magnetic wire according to a preferred embodiment of the present invention;

FIG. 5 depicts a waveform delivered to a target pathway structure such as a molecule, cell, tissue or organ according to a preferred embodiment of the present invention;

FIG. 6 is a view of a positioning device such as a wrist support according to a preferred embodiment of the present invention;

FIG. 7 is a view of a positioning device such as a mattress pad according to a preferred embodiment of the present invention;

FIG. 8 is a view of a positioning device such as a chest garment according to an embodiment of the present invention;

FIG. 9 is a graph illustrating maximally increased myosin phosphorylation for a PMRF signal configured according to an embodiment of the present invention.

### DETAILED DESCRIPTION

An embodiment according to the present invention provides a higher spectral density to a pulse burst envelope resulting in enhanced effectiveness of therapy upon relevant dielectric pathways, such as, cellular membrane receptors, ion binding to cellular enzymes and general transmembrane potential changes. An embodiment according to the present invention increases the number of frequency components transmitted to relevant cellular pathways, thereby providing access to a larger range of biophysical phenomena applicable to known healing mechanisms, for example modulation of growth factor and cytokine release, and ion binding at regulatory molecules. By applying a random, or other high spectral density envelope, according to a mathematical model defined by SNR or Power SNR in a transduction pathway, to a pulse burst envelope of mono- or bi-polar rectangular or sinusoidal pulses inducing peak electric fields between 10⁻⁶ and 10 volts per centimeter (V/cm), a greater effect could be accomplished on biological healing processes applicable to both soft and hard tissues.

An advantageous result of the present invention, is that by applying a high spectral density voltage envelope as the modulating or pulse-burst defining parameter, according to a mathematical model defined by SNR or Power SNR in a transduction pathway, the power requirement for such amplitude modulated pulse bursts can be significantly lower than that of an unmodulated pulse burst containing pulses within the same frequency range. Accordingly, the advantages of enhanced transmitted dosimetry to the relevant dielectric target pathways and of decreased power requirement are achieved. Another advantage of the present invention is the acceleration of wound repair.

Known mechanisms of wound repair involve the naturally timed release of the appropriate growth factor or cytokine in each stage of wound repair as applied to humans, animals and plants. Specifically, wound repair involves an inflammatory phase, angiogenesis, cell proliferation, collagen production, and remodeling stages. There are timed releases of specific cytokines and growth factors in each stage. Electromagnetic fields are known to enhance blood flow and to enhance the binding of ions which, in turn, can accelerate each healing phase. It is an object of this invention to provide an improved means to enhance the action and accelerate the intended effects or improve efficacy as well as other effects of the cytokines and growth factors relevant to each stage of wound repair.

Induced time-varying currents from PEMF or PRF devices flow in a target pathway structure such as a molecule, cell, tissue, and organ, and it is these currents that are a stimulus to which cells and tissues can react in a physiologically meaningful manner. The electrical properties of a target pathway structure affect levels and distributions of induced current. Molecules, cells, tissue, and organs are all in an induced current pathway such as cells in a gap junction contact. Ion or ligand interactions at binding sites on macromolecules that may reside on a membrane surface are voltage dependent processes, for example electrochemical, that can respond to an induced electromagnetic field (“E”). Induced current arrives at these sites via a surrounding ionic medium. The presence of cells in a current pathway causes an induced current (“J”) to decay more rapidly with time (“J(t)”). This is due to an added electrical impedance of cells from membrane capacitance and time constants of binding and other voltage sensitive membrane processes such as membrane transport.

Equivalent electrical circuit models representing various membrane and charged interface configurations have been derived. For example, in Calcium (“Ca²+”) binding, the change in concentration of bound Ca²+at a binding site due to induced E may be described in a frequency domain by an impedance expression such as:

$${Z}_{b} ( ω ) = {R}_{ion} + \frac{1}{{ⅈωC}_{ion}}$$

which has the form of a series resistance-capacitance electrical equivalent circuit. Where ω is angular frequency defined as 2πf, where f is frequency, i=−1^{1/2}, Z_{b}(ω) is the binding impedance, and Rᵢₒₙ and Cᵢₒₙ are equivalent binding resistance and capacitance of an ion binding pathway. The value of the equivalent binding time constant, τᵢₒₙ=RᵢₒₙCᵢₒₙ, is related to a ion binding rate constant, kb, via τᵢₒₙ=RᵢₒₙCᵢₒₙ=1/kb. Thus, the characteristic time constant of this pathway is determined by ion binding kinetics.

Induced E from a PEMF or PRF signal can cause current to flow into an ion binding pathway and affect the number of Ca²⁺ ions bound per unit time. An electrical equivalent of this is a change in voltage across the equivalent binding capacitance Cᵢₒₙ, which is a direct measure of the change in electrical charge stored by Cᵢₒₙ. Electrical charge is directly proportional to a surface concentration of Ca²⁺ions in the binding site, that is storage of charge is equivalent to storage of ions or other charged species on cell surfaces and junctions. Electrical impedance measurements, as well as direct kinetic analyses of binding rate constants, provide values for time constants necessary for configuration of a PMF waveform to match a bandpass of target pathway structures. This allows for a required range of frequencies for any given induced E waveform for optimal coupling to target impedance, such as bandpass.

Ion binding to regulatory molecules is a frequent EMF target, for example Ca²⁺binding to calmodulin (“CaM”). Use of this pathway is based upon acceleration of tissue repair, for example bone repair, wound repair, hair repair, and repair of molecules, cells, tissues, and organs that involves modulation of growth factors released in various stages of repair. Growth factors such as platelet derived growth factor (“PDGF”), fibroblast growth factor (“FGF”), and epidermal growth factor (“EGF”) are all involved at an appropriate stage of healing. Angiogenesis and neovascularization are also integral to tissue growth and repair and can be modulated by PMF. All of these factors are Ca/CaM-dependent.

Utilizing a Ca/CaM pathway a waveform can be configured for which induced power is sufficiently above background thermal noise power. Under correct physiological conditions, this waveform can have a physiologically significant bioeffect.

Application of a Power SNR model to Ca/CaM requires knowledge of electrical equivalents of Ca²+binding kinetics at CaM. Within first order binding kinetics, changes in concentration of bound Ca²⁺at CaM binding sites over time may be characterized in a frequency domain by an equivalent binding time constant, τᵢₒₙ=RᵢₒₙCᵢₒₙ, where Rᵢₒₙ and Cᵢₒₙ are equivalent binding resistance and capacitance of the ion binding pathway. τᵢₒₙ is related to a ion binding rate constant, k_{b}, via τᵢₒₙ=RᵢₒₙCᵢₒₙ=1/k_{b}. Published values for k_{b} can then be employed in a cell array model to evaluate SNR by comparing voltage induced by a PRF signal to thermal fluctuations in voltage at a CaM binding site. Employing numerical values for PMF response, such as Vₘₐₓ=6.5×10⁻⁷ sec₋₁, [Ca²⁺]=2.5μM, K_{D}=30μM, [Ca²⁺CaM]=K_{D}([Ca²⁺]+[CaM]), yields k_{b}=665 sec⁻¹ (τᵢₒₙ=1.5 msec). Such a value for τᵢₒₙ can be employed in an electrical equivalent circuit for ion binding while power SNR analysis can be performed for any waveform structure.

According to an embodiment of the present invention a mathematical model for example a mathematical equation and or a series of mathematical equations can be configured to assimilate that thermal noise is present in all voltage dependent processes and represents a minimum threshold requirement to establish adequate SNR. For example a mathematical model that represents a minimum threshold requirement to establish adequate SNR can be configured to include power spectral density of thermal noise such that power spectral density, Sₙ(ω), of thermal noise can be expressed as:
Sₙ(ω)=4kT Re[Zₘ(x,ω)]
where Z_{M}(x,ω) is electrical impedance of a target pathway structure, x is a dimension of a target pathway structure and Re denotes a real part of impedance of a target pathway structure. Z_{M}(x,ω) can be expressed as:

$${Z}_{M} ( x , ω ) = [ \frac{{R}_{e} + {R}_{i} + {R}_{g}}{γ} ] tanh ( γ x )$$

This equation clearly shows that electrical impedance of the target pathway structure, and contributions from extracellular fluid resistance (“Rₑ”), intracellular fluid resistance (“Rᵢ”) and intermembrane resistance (“R_{g}”) which are electrically connected to a target pathway structures, all contribute to noise filtering.

A typical approach to evaluation of SNR uses a single value of a root mean square (RMS) noise voltage. This is calculated by taking a square root of an integration of Sₙ(ω)=4kT Re[Zₘ(x,ω)] over all frequencies relevant to either complete membrane response, or to bandwidth of a target pathway structure. SNR can be expressed by a ratio:

$$SNR = \frac{\vert {V}_{M} ( ω ) \vert}{RMS}$$

where |V_{M}(ω)| is maximum amplitude of voltage at each frequency as delivered by a chosen waveform to the target pathway structure.

An embodiment according to the present invention comprises a pulse burst envelope having a high spectral density, so that the effect of therapy upon the relevant dielectric pathways, such as, cellular membrane receptors, ion binding to cellular enzymes and general transmembrane potential changes, is enhanced. Accordingly by increasing a number of frequency components transmitted to relevant cellular pathways, a large range of biophysical phenomena, such as modulating growth factor and cytokine release and ion binding at regulatory molecules, applicable to known tissue growth mechanisms is accessible. According to an embodiment of the present invention applying a random, or other high spectral density envelope, to a pulse burst envelope of mono- or bi-polar rectangular or sinusoidal pulses inducing peak electric fields between about 10₋₈ and about 100 V/cm, produces a greater effect on biological healing processes applicable to both soft and hard tissues.

According to yet another embodiment of the present invention by applying a high spectral density voltage envelope as a modulating or pulse-burst defining parameter, power requirements for such amplitude modulated pulse bursts can be significantly lower than that of an unmodulated pulse burst containing pulses within a similar frequency range. This is due to a substantial reduction in duty cycle within repetitive burst trains brought about by imposition of an irregular, and preferably random, amplitude onto what would otherwise be a substantially uniform pulse burst envelope. Accordingly, the dual advantages, of enhanced transmitted dosimetry to the relevant dielectric pathways and of decreased power requirement are achieved.

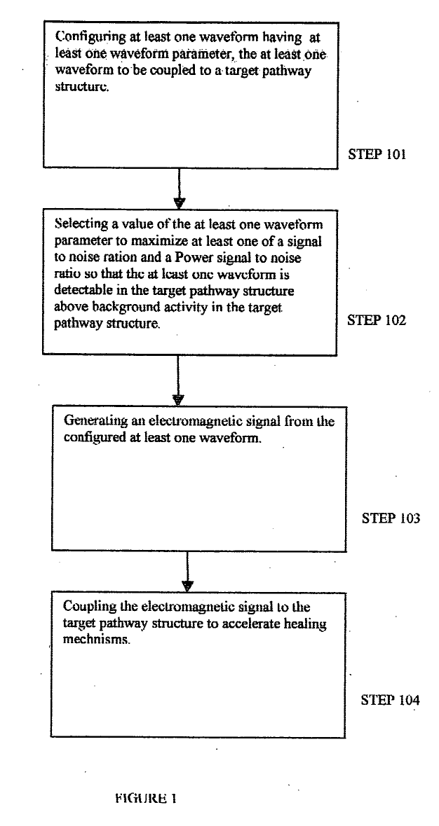

Referring to FIG. 1, wherein FIG. 1 is a flow diagram of a method according to an embodiment of the present invention, for accelerating wound repair by delivering electromagnetic signals that can be pulsed, to target pathway structures such as ions and ligands of animals and humans, for therapeutic and prophylactic purposes. Target pathway structures can also include but are not limited to tissues, cells, organs, and molecules.

Configuring at least one waveform having at least one waveform parameter to be coupled to the target pathway structure such as ions and ligands (Step 101).

The at least one waveform parameter is selected to maximize at least one of a signal to noise ratio and a Power Signal to Noise ratio in a target pathway structure so that a waveform is detectable in the target pathway structure above its background activity (Step 102) such as baseline thermal fluctuations in voltage and electrical impedance at a target pathway structure that depend upon a state of a cell and tissue, that is whether the state is at least one of resting, growing, replacing, and responding to injury to produce physiologically beneficial results. To be detectable in the target pathway structure the value of said at least one waveform parameter is chosen by using a constant of said target pathway structure to evaluate at least one of a signal to noise ratio, and a Power signal to noise ratio, to compare voltage induced by said at least one waveform in said target pathway structure to baseline thermal fluctuations in voltage and electrical impedance in said target pathway structure whereby bioeffective modulation occurs in said target pathway structure by said at least one waveform by maximizing said at least one of signal to noise ratio and Power signal to noise ratio, within a bandpass of said target pathway structure.

A preferred embodiment of a generated electromagnetic signal is comprised of a burst of arbitrary waveforms having at least one waveform parameter that includes a plurality of frequency components ranging from about 0.01 Hz to about 100 MHz wherein the plurality of frequency components satisfies a Power SNR model (Step 103). A repetitive electromagnetic signal can be generated for example inductively or capacitively, from said configured at least one waveform (Step 104). The electromagnetic signal can also be non-repetitive. The electromagnetic signal is coupled to a target pathway structure such as ions and ligands by output of a coupling device such as an electrode or an inductor, placed in close proximity to the target pathway structure (Step 105). The coupling enhances blood flow and modulation of binding of ions and ligands to regulatory molecules in molecules, tissues, cells, and organs thereby accelerating wound repair.

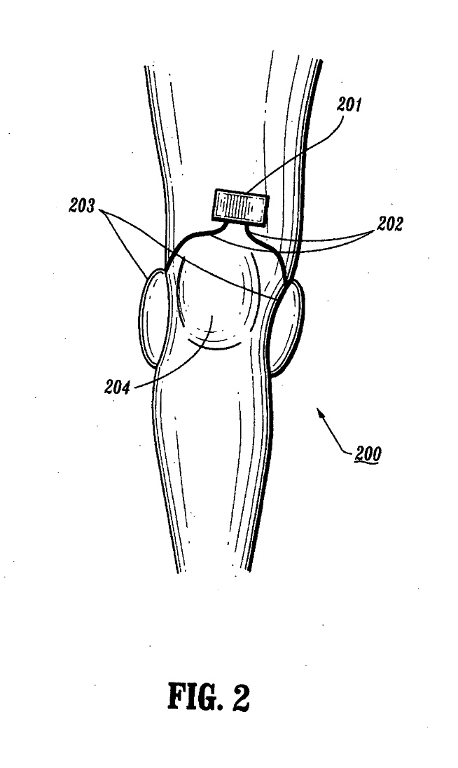

FIG. 2 illustrates a preferred embodiment of an apparatus according to the present invention. The apparatus is self-contained, lightweight, and portable. A miniature control circuit 201 is coupled to an end of at least one connector 202 such as wire however the control circuit can also operate wirelessly. The opposite end of the at least one connector is coupled to a generating device such as an electrical coil 203. The miniature control circuit 201 is constructed in a manner that applies a mathematical model that is used to configure waveforms. The configured waveforms have to satisfy Power SNR so that for a given and known target pathway structure, it is possible to choose waveform parameters that satisfy Power SNR so that a waveform produces physiologically beneficial results, for example bioeffective modulation, and is detectable in the target pathway structure above its background activity. A preferred embodiment according to the present invention applies a mathematical model to induce a time-varying magnetic field and a time-varying electric field in a target pathway structure such as ions and ligands, comprising about 0.1 to about 100 msec bursts of about 1 to about 100 microsecond rectangular pulses repeating at about 0.1 to about 100 pulses per second. Peak amplitude of the induced electric field is between about 1 uV/cm and about 100 mV/cm, varied according to a modified 1/f function where f=frequency. A waveform configured using a preferred embodiment according to the present invention may be applied to a target pathway structure such as ions and ligands for a preferred total exposure time of under 1 minute to 240 minutes daily. However other exposure times can be used. Waveforms configured by the miniature control circuit 201 are directed to a generating device 203 such as electrical coils via connector 202. The generating device 203 delivers a pulsing magnetic field that can be used to provide treatment to a target pathway structure such as tissue. The miniature control circuit applies a pulsing magnetic field for a prescribed time and can automatically repeat applying the pulsing magnetic field for as many applications as are needed in a given time period, for example 10 times a day. The miniature control circuit can be configured to be programmable applying pulsing magnetic fields for any time repetition sequence. A preferred embodiment according to the present invention can accelerate wound repair by being incorporated into a positioning device 204, for example a bed. Coupling a pulsing magnetic field to a target pathway structure such as ions and ligands, therapeutically and prophylactically reduces inflammation thereby advantageously reducing pain, promoting healing in targeted areas. When electrical coils are used as the generating device 203, the electrical coils can be powered with a time varying magnetic field that induces a time varying electric field in a target pathway structure according to Faraday's law. An electromagnetic signal generated by the generating device 203 can also be applied using electrochemical coupling, wherein electrodes are in direct contact with skin or another outer electrically conductive boundary of a target pathway structure. Yet in another embodiment according to the present invention, the electromagnetic signal generated by the generating device 203 can also be applied using electrostatic coupling wherein an air gap exists between a generating device 203 such as an electrode and a target pathway structure such as ions and ligands. An advantage of the preferred embodiment according to the present invention is that its ultra lightweight coils and miniaturized circuitry allow for use with common physical therapy treatment modalities and at any for which growth, pain relief, and tissue and organ healing is desired. An advantageous result of application of the preferred embodiment according to the present invention is that tissue growth, repair, and maintenance can be accomplished and enhanced anywhere and at anytime, for example while driving a car or watching television. Yet another advantageous result of application of the preferred embodiment is that growth, repair, and maintenance of molecules, cells, tissues, and organs can be accomplished and enhanced anywhere and at anytime, for example while driving a car or watching television.

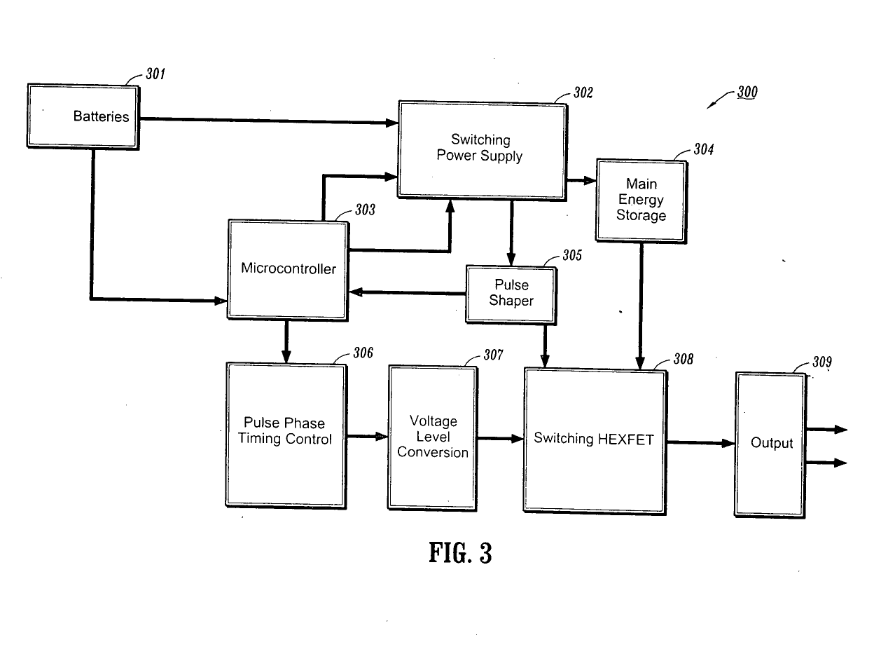

FIG. 3 depicts a block diagram of a preferred embodiment according to the present invention of a miniature control circuit 300. The miniature control circuit 300 produces waveforms that drive a generating device such as wire coils described above in FIG. 2. The miniature control circuit can be activated by any activation means such as an on/off switch. The miniature control circuit 300 has a power source such as a lithium battery 301. A preferred embodiment of the power source has an output voltage of 3.3 V but other voltages can be used. In another embodiment according to the present invention the power source can be an external power source such as an electric current outlet such as an AC/DC outlet, coupled to the present invention for example by a plug and wire. A switching power supply 302 controls voltage to a micro-controller 303. A preferred embodiment of the micro-controller 303 uses an 8 bit 4 MHz micro-controller 303 but other bit MHz combination micro-controllers may be used. The switching power supply 302 also delivers current to storage capacitors 304. A preferred embodiment of the present invention uses storage capacitors having a 220 uF output but other outputs can be used. The storage capacitors 304 allow high frequency pulses to be delivered to a coupling device such as inductors (Not Shown). The micro-controller 303 also controls a pulse shaper 305 and a pulse phase timing control 306. The pulse shaper 305 and pulse phase timing control 306 determine pulse shape, burst width, burst envelope shape, and burst repetition rate. An integral waveform generator, such as a sine wave or arbitrary number generator can also be incorporated to provide specific waveforms. A voltage level conversion sub-circuit 307 controls an induced field delivered to a target pathway structure. A switching Hexfet 308 allows pulses of randomized amplitude to be delivered to output 309 that routes a waveform to at least one coupling device such as an inductor. The micro-controller 303 can also control total exposure time of a single treatment of a target pathway structure such as a molecule, cell, tissue, and organ. The miniature control circuit 300 can be constructed to be programmable and apply a pulsing magnetic field for a prescribed time and to automatically repeat applying the pulsing magnetic field for as many applications as are needed in a given time period, for example 10 times a day. A preferred embodiment according to the present invention uses treatments times of about 10 minutes to about 30 minutes.

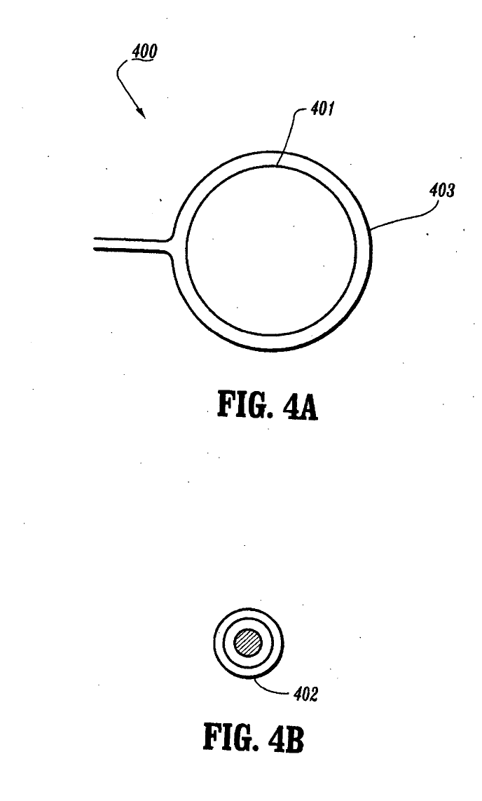

Referring to FIGS. 4A and 4B a preferred embodiment according to the present invention of a coupling device 400 such as an inductor is shown. The coupling device 400 can be an electric coil 401 wound with single or multistrand flexible wire 402 however solid wire can also be used. In a preferred embodiment according to the present invention the wire is made of copper but other materials can be used. The multistrand flexible magnetic wire 402 enables the electric coil 401 to conform to specific anatomical configurations such as a limb or joint of a human or animal. A preferred embodiment of the electric coil 401 comprises about 1 to about 1000 turns of about 0.01 mm to about 0.1 mm diameter at least one of single magnet wire and multistrand magnet wire, wound on an initially circular form having an outer diameter between about 2.5 cm and about 50 cm but other numbers of turns and wire diameters can be used. A preferred embodiment of the electric coil 401 can be encased with a non-toxic PVC mould 403 but other non-toxic moulds can also be used. The electric coil can also be incorporated in dressings, bandages, garments, and other structures typically used for wound treatment.

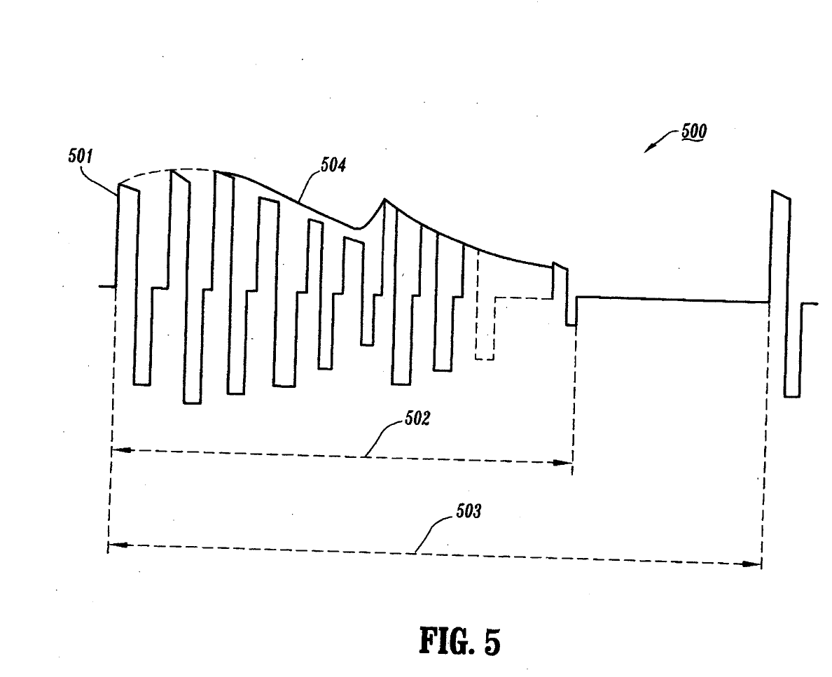

Referring to FIG. 5 an embodiment according to the present invention of a waveform 500 is illustrated. A pulse 501 is repeated within a burst 502 that has a finite duration 503. The duration 503 is such that a duty cycle which can be defined as a ratio of burst duration to signal period is between about 1 to about 10₋₅. A preferred embodiment according to the present invention utilizes pseudo rectangular 10 microsecond pulses for pulse 501 applied in a burst 502 for about 10 to about 50 msec having a modified 1/f amplitude envelope 504 and with a finite duration 503 corresponding to a burst period of between about 0.1 and about 10 seconds but other waveforms, envelopes, and burst periods may be used that conform to a mathematical model such as SNR and Power SNR.

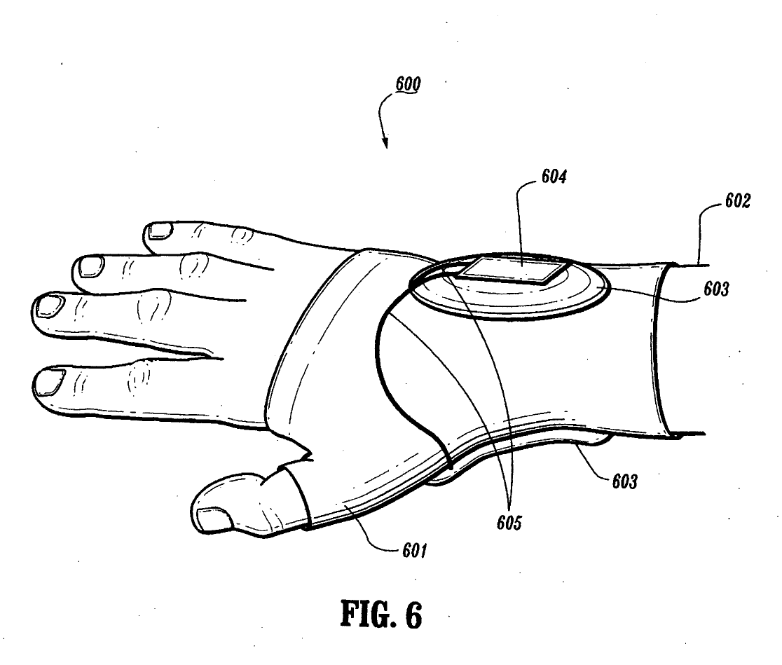

FIG. 6 illustrates a preferred embodiment according to the present invention of a positioning device such as a wrist support. A positioning device 600 such as a wrist support 601 is worn on a human wrist 602. The positioning device can be constructed to be portable, can be constructed to be disposable, and can be constructed to be implantable. The positioning device can be used in combination with the present invention in a plurality of ways, for example incorporating the present invention into the positioning device for example by stitching, affixing the present invention onto the positioning device for example by Velcro®, and holding the present invention in place by constructing the positioning device to be elastic.

In another embodiment according to the present invention, the present invention can be constructed as a stand-alone device of any size with or without a positioning device, to be used anywhere for example at home, at a clinic, at a treatment center, and outdoors. The wrist support 601 can be made with any anatomical and support material, such as neoprene. Coils 603 are integrated into the wrist support 601 such that a signal configured according to the present invention, for example the waveform depicted in FIG. 5, is applied from a dorsal portion that is the top of the wrist to a plantar portion that is the bottom of the wrist. Micro-circuitry 604 is attached to the exterior of the wrist support 601 using a fastening device such as Velcro® (Not Shown). The micro-circuitry is coupled to one end of at least one connecting device such as a flexible wire 605. The other end of the at least one connecting device is coupled to the coils 603. Other embodiments according to the present invention of the positioning device include knee, elbow, lower back, shoulder, other anatomical wraps, and apparel such as garments, fashion accessories, and footware.

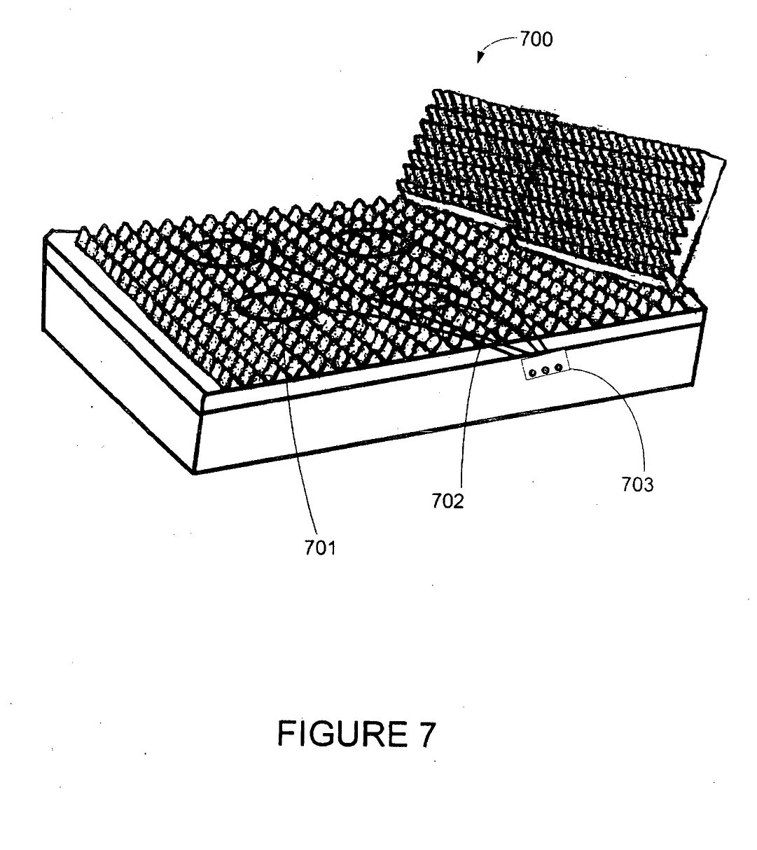

Referring to FIG. 7 an embodiment according to the present invention of an electromagnetic treatment apparatus integrated into a mattress pad 700 is illustrated. A mattress can also be used. Several lightweight flexible coils 701 are integrated into the mattress pad. The lightweight flexible coils can be constructed from fine flexible conductive wire, conductive thread, and any other flexible conductive material. The flexible coils are connected to at least one end of at least one wire 702. However, the flexible coils can also be configured to be directly connected to circuitry 703 or wireless. Lightweight miniaturized circuitry 703 that configures waveforms according to an embodiment of the present invention, is attached to at least one other end of said at least on wire. When activated the lightweight miniaturized circuitry 703 configures waveforms that are directed to the flexible coils (701) to create PEMF signals that are coupled to a target pathway structure.

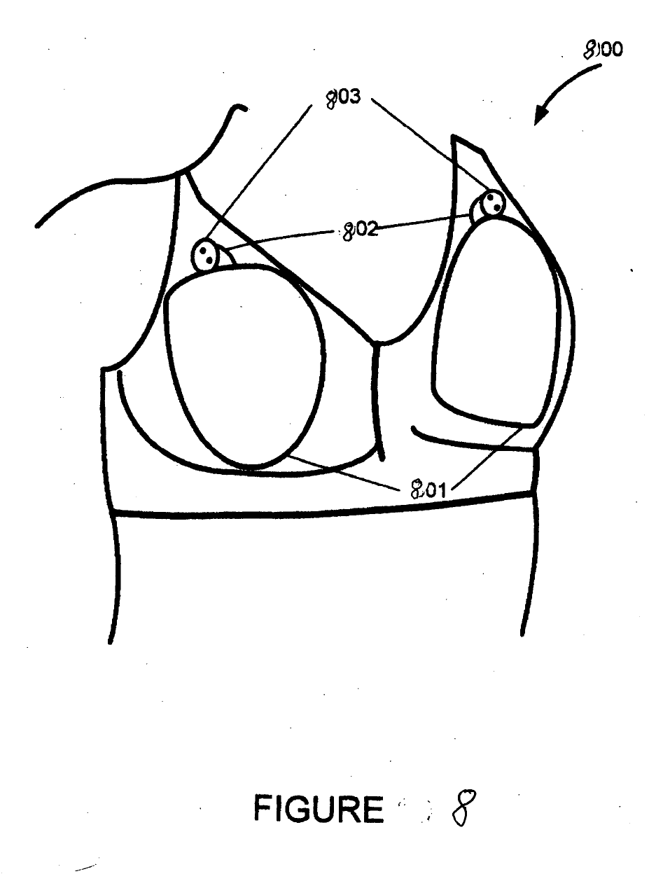

Referring to FIG. 8 an embodiment according to the present invention of an electromagnetic treatment inductive apparatus integrated into a chest garment 800, such as a bra is illustrated. Several lightweight flexible coils 801 are integrated into a bra. The lightweight flexible coils can be constructed from fine flexible conductive wire, conductive thread, and any other flexible conductive material. The flexible coils are connected to at least one end of at least one wire 802. However, the flexible coils can also be configured to be directly connected to circuitry 803 or wireless. Lightweight miniaturized circuitry 803 that configures waveforms according to an embodiment of the present invention, is attached to at least one other end of said at least on wire. When activated the lightweight miniaturized circuitry 803 configures waveforms that are directed to the flexible coils (801) to create PEMF signals that are coupled to a target pathway structure.

### EXAMPLE 1

An embodiment according to the present invention for EMF signal configuration has been used on calcium dependent myosin phosphorylation in a standard enzyme assay. This enzyme pathway is known to enhance the effects of pharmacological, chemical, cosmetic and topical agents as applied to, upon or in human, animal and plant cells, organs, tissues and molecules. The reaction mixture was chosen for phosphorylation rate to be linear in time for several minutes, and for sub-saturation Ca²⁺concentration. This opens the biological window for Ca²⁺/CaM to be EMF-sensitive, as happens in an injury or with the application of pharmacological, chemical, cosmetic and topical agents as applied to, upon or in human, animal and plant cells, organs, tissues and molecules. Experiments were performed using myosin light chain (“MLC”) and myosin light chain kinase (“MLCK”) isolated from turkey gizzard. A reaction mixture consisted of a basic solution containing 40 mM Hepes buffer, pH 7.0; 0.5 mM magnesium acetate; 1 mg/ml bovine serum albumin, 0.1% (w/v) Tween 80; and 1 mM EGTA. Free Ca²⁺was varied in the 1-7 μM range. Once Ca²⁺buffering was established, freshly prepared 70 nM CaM, 160 nM MLC and 2 nM MLCK were added to the basic solution to form a final reaction mixture.

The reaction mixture was freshly prepared daily for each series of experiments and was aliquoted in 100 μL portions into 1.5 ml Eppendorf tubes. All Eppendorf tubes containing reaction mixture were kept at 0° C. then transferred to a specially designed water bath maintained at 37±0.1° C. by constant perfusion of water prewarmed by passage through a Fisher Scientific model 900 heat exchanger. Temperature was monitored with a thermistor probe such as a Cole-Parmer model 8110-20, immersed in one Eppendorf tube during all experiments. Reaction was initiated with 2.5 μM 32P ATP, and was stopped with Laemmli Sample Buffer solution containing 30 μM EDTA. A minimum of five blank samples were counted in each experiment. Blanks comprised a total assay mixture minus one of the active components Ca²⁺, CaM, MLC or MLCK. Experiments for which blank counts were higher than 300 μm were rejected. Phosphorylation was allowed to proceed for 5 min and was evaluated by counting ³²p incorporated in MLC using a TM Analytic model 5303 Mark V liquid scintillation counter.

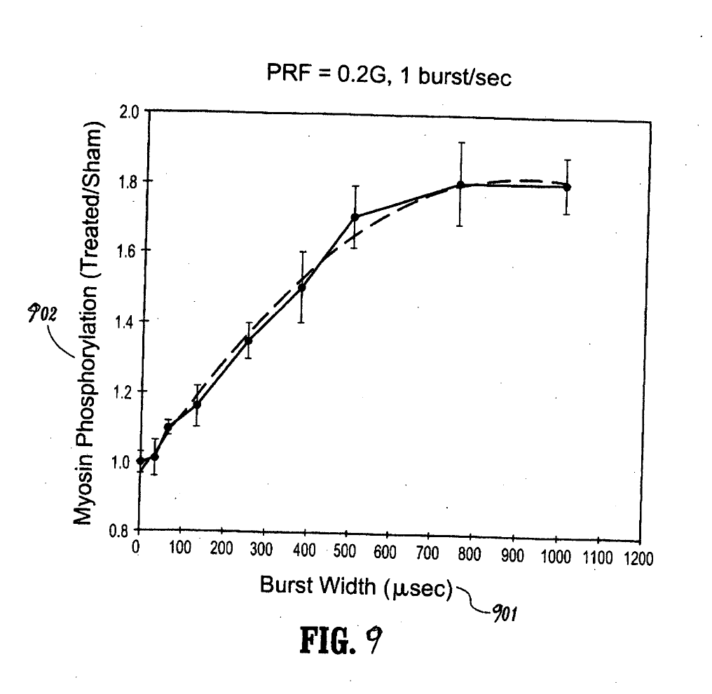

The signal comprised repetitive bursts of a high frequency waveform. Amplitude was maintained constant at 0.2G and repetition rate was 1 burst/sec for all exposures. Burst duration varied from 65 psec to 1000 psec based upon projections of mathematical analysis of the instant invention which showed that optimal Power SNR would be achieved as burst duration approached 500 μsec. The results are shown in FIG. 9 wherein burst width 901 in μsec is plotted on the x-axis and Myosin Phosphorylation 902 as treated/sham is plotted on the y-axis. It can be seen that the PMF effect on Ca²⁺binding to CaM approaches its maximum at approximately 500 μsec, just as illustrated by the Power SNR model.

These results confirm that an EMF signal, configured according to an embodiment of the present invention, would maximally increase wound repair in human, animal and plant cells, organs, tissues and molecules for burst durations sufficient to achieve optimal Power SNR for a given magnetic field amplitude.

### EXAMPLE 2

According to an embodiment of the present invention use of a Power SNR model was further verified in an in vivo wound repair model. A rat wound model has been well characterized both biomechanically and biochemically, and was used in this study. Healthy, young adult male Sprague Dawley rats weighing more than 300 grams were utilized.

The animals were anesthetized with an intraperitoneal dose of Ketamine 75 mg/kg and Medetomidine 0.5 mg/kg. After adequate anesthesia had been achieved, the dorsum was shaved, prepped with a dilute betadine/alcohol solution, and draped using sterile technique. Using a #10 scalpel, an 8-cm linear incision was performed through the skin down to the fascia on the dorsum of each rat. The wound edges were bluntly dissected to break any remaining dermal fibers, leaving an open wound approximately 4 cm in diameter.

Hemostasis was obtained with applied pressure to avoid any damage to the skin edges. The skin edges were then closed with a 4-0 Ethilon running suture. Post-operatively, the animals received Buprenorphine 0.1-0.5mg/kg, intraperitoneal. They were placed in individual cages and received food and water ad libitum.

EMF exposure comprised two pulsed radio frequency waveforms. The first was a standard clinical PRF signal comprising a 65 μsec burst of 27.12 MHz sinusoidal waves at 1 Gauss amplitude and repeating at 600 bursts/sec. The second was a PRF signal reconfigured according to an embodiment of the present invention. For this signal burst duration was increased to 2000 μsec and the amplitude and repetition rate were reduced to 0.2G and 5 bursts/sec respectively. PRF was applied for 30 minutes twice daily. Tensile strength was performed immediately after wound excision. Two 1 cm width strips of skin were transected perpendicular to the scar from each sample, and used to measure the tensile strength in kg/mm². The strips were excised from the same area in each rat to assure consistency of measurement. The strips were then mounted on a tensiometer. The strips were loaded at 10 mm/min and the maximum force generated before the wound pulled apart was recorded. The final tensile strength for comparison was determined by taking the average of the maximum load in kilograms per mm² of the two strips from the same wound. The results showed average tensile strength for the 65 μsec 1 Gauss PRF signal was 19.3±4.3 kg/mm² for the exposed group versus 13.0±3.5 kg/mm² for the control group (p<0.01), which is a 48% increase. In contrast, the average tensile strength for the 2000 μsec 0.2 Gauss PRF signal, configured according to an embodiment of the present invention using a Power SNR model was 21.2±5.6 kg/mm² for the treated group versus 13.7±4.1 kg/mm² (p<0.01) for the control group, which is a 54% increase. The results for the two signals were not significantly different from each other.

Non-invasive, non-thermal pulsed magnetic fields are successful therapies for healing non-union fractures, the palliative relief of pain and edema and the healing of chronic wounds. The two radio frequency EMF devices used in this study differed by burst duration, envelope, amplitude and repetition rate. That second radio frequency produced nearly identical results to those produced by first radio frequency demonstrates the validity of the EMF signal configuration according to the present invention.

The results follow the pattern observed in clinical and basic EMF studies. Applying correct dosimetry, that is the signal is detectable in the EMF-sensitive pathway, the state of the target determines the degree of effect. Thus, surrounding normal bone does not respond in a physiologically significant manner even though it receives the same EMF dosage as cells/tissue in the fracture site. The same occurs for cells in culture wherein a dependence upon cell cycle, state of tissue repair and the extracellular concentration of ions/ligands has been reported. Thus EMF has virtually no effect in the later stages of wound repair. By comparison with known biomechanical healing curve for this model, it may be estimated that the EMF treated wounds would have reached the end stage of wound repair, approximately 1.5×faster than the sham group.

At the cellular level PMF have been shown to enhance TGF-βproduction. EMF of the type used for bone repair significantly increased endothelial cell tubulization and proliferation, as well as fibroblast growth factor β-2, in vitro. Additionally, EMF signals can modulate anti-CD3 binding at lymphocyte receptors, demonstrating EMF can reduce the inflammatory response. When EMF effects occur in this cutaneous wound model, accelerated healing would be achieved, both from a reduction of time in the inflammatory phase and subsequent acceleration of collagen production. The production of growth factors has been reported to be Ca/CaM (calmodulin) dependent and an EMF signal has been shown to accelerate Ca2+binding to calmodulin. The electric field induced at tissue level from the EMF signal utilized has been shown to contain the proper frequency spectrum to be detected at Ca/CaM binding pathways. It has also been demonstrated that inductively coupled EMF bone healing signals can increase osteoblast proliferation in-vitro by direct modulation of Ca/CaM.

These results demonstrate that an embodiment of the present invention allowed a EMF signal to be configured that could be produced with significantly lower power. The PRF signal configured according to an embodiment of the present invention, accelerated wound repair in the rat model in a low power manner versus that for a clinical EMF signal which accelerated wound repair but required more than two orders of magnitude more power to produce.

### EXAMPLE 3

This study demonstrated the effect of electromagnetic fields configured according an embodiment of the present invention accelerate tendon repair in an in-vivo model.

Young adult male Sprague-Dawley rats, with a mean weight of 350 g, were anesthetized with an intraperitoneal injection of a ketamine/medetomidine 75 mg/kg/0.5 mg/kg mixture. The Achilles tendon was disrupted and repaired. Using sterile surgical technique, a 2-cm midline longitudinal incision was made over the right Achilles tendon while it was stretched by flexing the right foot. Blunt dissection was used to separate the tendon from the surrounding tissue, which was then transected at the middle using a scalpel. The Achilles tendon was then immediately repaired with 6-0 nylon suture using a modified Kessler stitch. The plantaris tendon was divided and not repaired. The skin was sutured over the repaired tendon using interrupted 5-0 Ethilon. The Achilles tendon was not immobilized. Postoperatively, the animals received Ketoprofen for pain control.

On the first postoperative day, all animals were randomly assigned to four treatment groups with 10 animals in each group. Randomization followed the parallel group protocol wherein each animal was randomly assigned to one treatment group until there were ten in each group. Animals remained in their assigned group. There were three active groups that received specific EMF treatments for two 30-min sessions per day over a period of 3 weeks, and one identically treated sham group. The EMF employed in this study was a pulsed radio frequency waveform comprising a repetitive burst of 27.12 MHz sinusoidal waves emitted by a PMF-generating coil. Two configurations were employed. The first, assigned to Group 1, comprised a burst duration of 65 psec, repeating at 600 bursts/sec with an amplitude at the tendon target of 1 gauss (“G”). The second PRF waveform comprised a burst duration of 2000 μsec, repeating at 5 bursts/sec with an amplitude at the tendon target of 0.05 G, assigned to Group 2, and 0.1 G, assigned to Group 3. Sham animals, no signal, were assigned to Group 4.

The PRF signal was delivered with a single loop coil, mounted to enable a standard rat plastic cage, with all metal portions removed, to be positioned within it. The coil was located 3.5 inches above, and horizontal to, the floor of the cage. Five freely roaming animals were treated with each coil. EMF signal amplitude was checked. Signal amplitude within the rat treatment cage over the normal range of rat movement was uniform to ±10%. Signal consistency was verified weekly. There were two cages each for the sham and active groups, and each cage had its individual coded EMF exposure system. EMF treatment was carried out twice daily for 30-min sessions until sacrifice. Sham animals were treated in identical cages equipped with identical coils.

At the end of the 3-week treatment period, the Achilles tendon was harvested by proximally severing the muscle bellies arising from the tendon and distally disarticulating the ankle, keeping the calcaneous and foot attached. All extraneous soft and hard tissues were removed from the calcaneous-Achilles tendon complex. Tensile strength testing was done immediately after harvest. The tendon, in continuity with the calcaneal bone, was fixed between two metal clamps so as to maintain a physiologically appropriate foot dorsiflexion, compared to the vertically oriented Achilles tendon. The tendons were then pulled apart at a constant speed of 0.45 mm/sec until failure, and the peak tensile strength was recorded. All analyzable tendons failed at the original transection. The tensile strengths from a total of 38 tendons were available for analysis.

Mean tensile strength was compared for each group at 3 weeks post tendon transection and data were analyzed. Tensile strength was calculated as the maximum breaking strength in kilograms per cross-sectional area in square centimeters. Tendons treated with the 65 μsec signal in Group 1 had a mean breaking strength of 99.4±14.6 kg/cm2 compared to 80.6±16.6 kg/cm2 for the sham-treated group in Group 4. This represented a 24% increase in breaking strength vs. the sham group at 21 days, which was not statistically significant (p=0.055). Tendons from Groups 2 and 3, treated with the 2000 μsec signals, had significantly higher mean breaking strengths of 129.4±27.8 kg/cm2 and 136.4±31.6 kg/cm2 for the 0.05 G and 0.1 G signals, respectively, vs. the sham exposure group 80.6 ±16.6 kg/cm2. The mean strengths for both Groups 2 and 3 were 60% and 69% higher, respectively, at the end of 3 weeks of treatment, compared to the sham group. This increase in strength was statistically significant (p<0.001); however, the difference in mean tensile strength between Groups 2 and 3 was not statistically significant (p=0.541). The differences in mean tensile strength between Group 1 (65 μsec burst) and Groups 2 and 3 (2000 μsec burst) was statistically significant (p<0.05).

The results presented here demonstrate that non-invasive pulsed electromagnetic fields can produce up to a 69% increase in rat Achilles tendon breaking strength vs. sham-treated tendons at 21 days post transection. All signals utilized in this study accelerated tendon repair, however greatest acceleration was obtained with waveforms configured according to a transduction mechanism involving Ca2+binding.

In a manner similar to bone and wound repair, tendon repair for both epitenon and synovial-sheathed tendons begins with an inflammatory stage that generally involves infiltration of inflammatory cells such as macrophages, neutrophils, and T-lymphocytes. This is followed by angiogenesis, fibroblast proliferation, and collagen mainly type III, production. Finally, cells and collagen fibrils orient to achieve maximum mechanical strength. These phases all occur in bone and wound repair, in which EMF has demonstrated effects, particularly in inflammatory, angiogenesis, and cell proliferation stages.

An EMF transduction pathway involves ion binding in regulatory pathways involving growth factor release. Production of many of the growth factors and cytokines involved in tissue growth and repair is dependent on Ca/CaM calmodulin. EMF has been shown to accelerate Ca2+binding to calmodulin. The 0.05 and 0.1 G signals utilized in this study were configured using a Ca/CaM transduction pathway. The objective was to produce sufficient electric field amplitude that is dose, within the frequency response of Ca2+binding. This would result in a lower power, more effective signal. The model demonstrated that microsecond range burst durations satisfy these objectives at amplitudes in the 0.05 G range. The 0.1 G signal was added to assure that the small size of the rat tendon target did hot limit the induced current pathway and reduce the expected dose.

EMF accelerates bone repair by accelerating return to intact breaking strength. The sham-treated fractures eventually reach the same biomechanical end point, but with increased morbidity. Biomechanical acceleration in a linear full-thickness cutaneous wound in the rat was observed. EMF accelerated wound repair by approximately 60% at 21 days, with intact breaking strength achieved about 50% sooner than the untreated wounds.

Having described embodiments for an apparatus and a method for enhancing pharmacological effects, it is noted that modifications and variations can be made by persons skilled in the art in light of the above teachings. It is therefore to be understood that changes may be made in the particular embodiments of the invention disclosed which are within the scope and spirit of the invention as defined by the appended claims.

What is claimed is:

1) A method for augmenting wound repair comprising the steps of:
Configuring at least one waveform having at least one waveform parameter;
Selecting a value of said at least one waveform parameter of said at least one waveform to maximize at least one of a signal to noise ratio and a Power signal to noise ratio, in a target pathway structure;
Using said at least one waveform that maximizes said at least one of a signal to noise ratio and a Power signal to noise ratio in a target pathway structure, to generate an electromagnetic signal; and
Coupling said electromagnetic signal to said target pathway structure to accelerate healing mechanisms.

2) The method of claim 1, wherein said healing mechanisms includes at least one of blood flow, neovascularization, vasodilatation, modulating human growth factors, and modulating angiogenesis.

3) The method of claim 1, wherein said at least one waveform parameter includes at least one of a frequency component parameter that configures said at least one waveform to repeat between about 0.01 Hz and about 100 MHz, a burst amplitude envelope parameter that follows a mathematically defined amplitude function, a burst width parameter that varies at each repetition according to a mathematically defined width function, a peak induced electric field parameter varying between about 1 μV/cm and about 100 mV/cm in said target pathway structure according to a mathematically defined function, and a peak induced magnetic electric field parameter varying between about 1 μT and about 0.1 T in said target pathway structure according to a mathematically defined function.

4) The method of claim 4, wherein said defined amplitude function includes at least one of a 1/frequency function, a logarithmic function, a chaotic function, and an exponential function.

5) The method of claim 1, wherein said target pathway structure includes at least one of molecules, cells, tissues, organs, ions, ligands, a chronic wound, diabetic ulcer, venous stasis ulcer, pressure sore, a non-healing wound, an acute wound, a post-surgical wound, and a post-trauma wound.

6) The method of claim 1, further comprising the step of binding ions and ligands to regulatory molecules to increase healing processes.

7) The method of claim 6, wherein said binding of ions and ligands includes modulating Calcium to Calmodulin binding.

8) The method of claim 6, wherein said binding of ions and ligands includes modulating growth factor production in target pathway structures.

9) The method of claim 6, wherein said binding of ions and ligands includes modulating cytokine production in target pathway structures.

10) The method of claim 6, wherein said binding of ions and ligands includes modulating growth factors and cytokines relevant to tissue growth, repair, and maintenance.

12) The method of claim 1, further comprising the step of applying of standard physical therapy modalities.

13) The method of claim 12, wherein standard physical therapy modalities includes at least one of heat, cold, compression, massage and exercise.

14) The method of claim 1, further comprising the step of applying of standard medical therapies.

15) The method of claim 14, wherein standard medical therapies includes at least one of tissue transplants and organ transplants.

16) An electromagnetic treatment apparatus for augmenting wound repair comprising:
A waveform production means that produces at least one waveform having at least one waveform parameter capable of being selected to maximize at least one of a signal to noise ratio and a Power signal to noise ratio in a target pathway structure while in a repair cycle; and
A coupling device connected to said waveform production means for generating an electromagnetic signal from said at least one waveform that maximizes said at least one of a signal to noise ratio and a Power signal to noise ratio in said target pathway structure, and for coupling said electromagnetic signal to said target pathway structure whereby said target pathway structure repair cycle is accelerated.

17) The electromagnetic treatment apparatus of claim 16, wherein said at least one waveform parameter includes at least one of a frequency component parameter that configures said at least one waveform to repeat between about 0.01 Hz and about 100 MHz according to a mathematical function, a burst amplitude envelope parameter that follows a mathematically defined amplitude function, a burst width parameter that varies at each repetition according to a mathematically defined width function, a peak induced electric field parameter varying between about 1 μV/cm and about 100 mV/cm in said target pathway structure according to a mathematically defined function, and a peak induced magnetic electric field parameter varying between about 1 μT and about 0.1 T in said target pathway structure according to a mathematically defined function.

18) The electromagnetic treatment apparatus of claim 17, wherein said defined amplitude function includes at least one of a 1/frequency function, a logarithmic function, a chaotic function, and an exponential function.

19) The electromagnetic treatment apparatus of claim 16, wherein said target pathway structure includes at least one of molecules, cells, tissues, organs, ions, ligands, a chronic wound, diabetic ulcer, venous stasis ulcer, pressure sore, a non-healing wound, an acute wound, a post-surgical wound, and a post-trauma wound.

20) The electromagnetic treatment apparatus of claim 16, wherein the coupling device includes at least one of a reactive coupling device, an inductive coupling device, a capacitive coupling device, and a biochemical coupling device.

21) The electromagnetic treatment apparatus of claim 16, wherein the coupling device couples said signal to said target pathway structure to modulate Calcium binding to Calmodulin.

22) The electromagnetic treatment apparatus of claim 16, wherein the coupling device couples said signal to said target pathway structure to modulate at least one of growth factor and cytokine production relevant.

23) The electromagnetic treatment apparatus of claim 22, wherein the growth factor includes at least one of fibroblast growth factors, platelet derived growth factors and interleukin growth factors.

24) The electromagnetic treatment apparatus of claim 16, wherein the coupling device couples said signal to said target pathway structure to modulate angiogenesis and neovascularization.

25) The electromagnetic treatment apparatus of claim 16, wherein the coupling device couples said signal to said target pathway structure to modulate human growth factor production.

26) The electromagnetic treatment apparatus of claim 16, wherein the coupling device said signal to said target pathway structure to augment cell and tissue activity.

27) The electromagnetic treatment apparatus of claim 16, wherein the coupling device couples said signal to said target pathway structure to increase cell population.

28) The electromagnetic treatment apparatus of claim 16, wherein the waveform production means, the connecting means, and the coupling device are configured to be lightweight, and portable.

29) The electromagnetic treatment apparatus of claim 16, wherein the waveform production means, the connecting means, and the coupling device are incorporated into at least one of a mattress, a mattress pad, a bed, and a positioning device.

30) The electromagnetic treatment apparatus of claim 29, wherein the positioning device includes at least one of an anatomical support, an anatomical wrap, and apparel.

31) The method of claim 30, wherein said apparel includes at least one of garments, fashion accessories, and footware.

32) The electromagnetic treatment apparatus of claim 16, wherein the waveform production means is programmable.

33) The electromagnetic treatment apparatus of claim 16, wherein the waveform production means delivers at least one pulsing magnetic signal during a predetermined time.

34) The electromagnetic treatment apparatus of claim 16, wherein the waveform production means delivers at least one pulsing magnetic signal during a random time.

35) The electromagnetic treatment apparatus of claim 16, further comprising a delivery means for standard physical therapy modalities.

36) The electromagnetic treatment apparatus of claim 35, wherein said standard physical therapy modalities includes heat, cold, massage, and exercise.
